(19) United States
(12) Patent Application Publication
(10) Pub. No.: US 2014/0172309 A1
(43) Pub. Date: Jun. 19, 2014

(54) CONTEXT AWARE BLOOD GLUCOSE MEASUREMENT SYSTEM

(71) Applicant: LIFESCAN SCOTLAND LIMITED, Inverness (GB)

(72) Inventor: Brian Guthrie, Inverness (GB)

(73) Assignee: LifeScan Scotland Limited, Inverness (GB)

(21) Appl. No.: 13/715,458

(22) Filed: Dec. 14, 2012

(51) Int. Cl. G01N 33/66; G01R 31/36; G06F 15/00

(52) U.S. Cl. CPC G01N 33/66; G06F 15/00; G01R 31/3606
USPC 702/19

(57) ABSTRACT

A blood glucose measurement system is configured to detect a wireless communication channel in proximity to the measurement system. An electronic data management unit of the measurement system automatically identifies a source of the detected wireless communication channel and uses the source information based on a stored rule set to automatically adjust at least one feature relating to the measurement system for use thereof.

### TECHNICAL FIELD

This application generally relates to the field of blood glucose measurement systems and more specifically to portable analyte meters that are configured for performing various functions based on user surroundings.

### BACKGROUND

Hand held blood glucose measurement systems are used for testing an individual's blood in a variety of surroundings at any time of day. These systems typically comprise an analyte meter that is configured to receive a biosensor, usually in the form of a test strip. Because these systems are portable, and testing can be completed in a short amount of time, patients are able to use such devices in the normal course of their daily lives without significant interruption to their personal routines. Therefore, a person with diabetes may measure their blood glucose levels several times a day as a part of a self management process to ensure glycemic control of their blood glucose within a target range. In the course of conducting typical day to day activities, the individual may perform a blood glucose test, for example, in a congested location such as an airport, subway or train station, while seated in a movie theater or while dining at a restaurant. With a bit of discretion, the individual can discreetly complete a blood glucose test so as not to bring unwanted attention to themselves.

A failure to maintain target glycemic control can result in serious diabetes-related complications including cardiovascular disease, kidney disease, nerve damage and blindness. The easier and more comfortable it is for an individual to perform blood glucose testing, the more likely that the person will be able to maintain target blood glucose levels. There currently exist a number of available portable electronic devices that can measure glucose levels in an individual based on a small sample of blood. One such analyte meter is the OneTouch® Verio™ glucose measurement system, a product which is manufactured by LifeScan, Inc.

### SUMMARY OF THE DISCLOSURE

Therefore and according to a first aspect, there is provided a blood glucose measurement system that includes but is not limited to an analyte meter and a biosensor, which is configured to detect a wireless communication channel in proximity to the analyte meter. An electronic data management unit disposed within the analyte meter automatically identifies a source of the detected wireless communication channel and uses that information to automatically adjust at least one feature relating to the analyte meter for enabling use thereof based on the identity of the source. One advantage that may be realized in the practice of some aspects disclosed herein of the blood glucose measurement system is that a user need not manually adjust output settings of the analyte meter in response to traveling to a new location before use because the meter automatically adjusts its output settings.

According to another aspect, an automated method of operating a wireless blood glucose measurement system which includes an analyte meter is disclosed. According to the method, a data management unit of the measurement system detects a wireless communication channel in proximity to the analyte meter and automatically identifies a source of the detected wireless communication channel. The data management unit automatically adjusts at least one feature relating to the measurement system for enabling use of the analyte meter based on the identity of the source.

In accordance with yet another aspect, an analyte meter may include a wireless communication circuit for receiving wireless communication transmitted from a nearby wireless communication source. The wireless communication includes a character string identifying a source of the wireless communication. A preloaded electronic table that includes but is not limited to a plurality of character strings is stored in association with information describing an identity of a corresponding wireless communication source. A data management unit may include a circuit for adjusting at least one of a visual or audio output level of the analyte meter based on the identity of the communication source.

In another aspect, a method of operating a portable wireless blood glucose measurement system may include detecting a wireless communication channel, determining parameters of a location of the measurement system based on the detected wireless communication channel, and adjusting a data output level of the measurement system based on the parameters of the location.

These and other embodiments, features and advantages will become apparent to those skilled in the art when taken with reference to the following more detailed modes of carrying out the invention in conjunction with the accompanying drawings that are first briefly described.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention (wherein like numerals represent like elements).

FIG. 1A illustrates a diagram of an exemplary blood glucose measurement system;

FIG. 1B illustrates a diagram of an exemplary data management unit of the blood glucose measurement system of FIG. 1A;

FIG. 2 illustrates a scenario wherein an analyte meter of the system of FIG. 1A is in proximity to an available wireless access point;

FIG. 3 illustrates a flow chart of an exemplary method of adjusting at least one feature of the blood glucose measurement system in response to information about the wireless access point;

FIG. 4 illustrates a flow chart of another exemplary method of adjusting at least one feature of the blood glucose measurement system in response to information about the wireless access point;

FIG. 5 illustrates a flow chart of a method of notifying a user in regard to an opportunity for obtaining supplies useful for maintaining operability of the blood glucose measurement system; and

FIG. 6 illustrates a flow chart of another method of operating the blood glucose measurement system in response to information about the wireless access point.

### MODES OF CARRYING OUT THE INVENTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “patient” or “user” refer to any human or animal subject and are not intended to limit the systems or methods to human use, although use of the subject invention in a human patient represents a preferred embodiment.

As used herein, the term “wireless access point” refers to a source of available wireless network access by a wireless device.

Unless indicated otherwise in the text, the term “location” refers to a type, context, or physical characteristics of a location, and not to absolute geographic location such as is identifiable by global positioning coordinates.

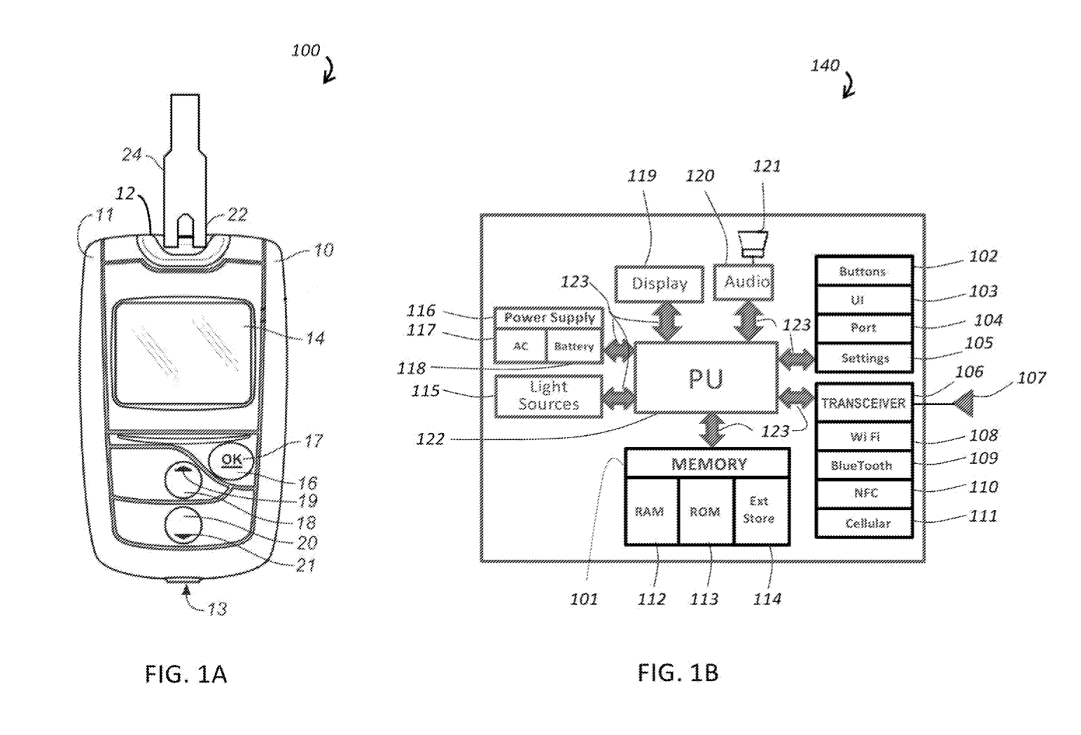

FIG. 1A illustrates an analyte measurement system 100 that includes an analyte meter 10. The analyte meter 10 is defined by a housing 11 that retains a data management unit 140 and further includes a port 22 sized for receiving a biosensor. According to this embodiment, the analyte meter 10 is a blood glucose meter and the biosensor is provided in the form of a glucose test strip 24 for performing blood glucose measurements. It is noted that while the biosensor 24 is shown in the form of a test strip to test blood glucose, a continuous glucose monitor can also be utilized as an alternative to the embodiments described herein. The analyte meter 10 includes a data management unit 140, FIG. 1B, disposed within the interior of the meter housing 11, a plurality of user interface buttons (16, 18, and 20), a display 14, a strip port connector 22, a strip port illumination panel 12, and a data port 13, as illustrated in FIG. 1A. A predetermined number of glucose test strips may be stored in the housing 11 and made accessible for use in blood glucose testing. The plurality of user interface buttons (16, 18, and 20) can be configured to allow the entry of data, to prompt an output of data, to navigate menus presented on the display 14, and to execute commands. Output data can include values representative of analyte concentration presented on the display 14. Input information, which are related to the everyday lifestyle of an individual, can include food intake, medication use, occurrence of health check-ups, and general health condition and exercise levels of an individual. These inputs can be requested via prompts presented on the display 14 and can be stored in a memory module of the analyte meter 10. Specifically and according to this exemplary embodiment, the user interface buttons 16, 18, and 20 include a first user interface button 16, a second user interface button 18, and a third user interface button 20. In that regard, the user interface buttons (16, 18, and 20) further include a first marking 17, a second marking 19, and a third marking 21, respectively, which allow a user to navigate through the user interface presented on the display 14. Although the buttons 16, 18, 20 are shown herein as mechanical switches, a touch screen interface with virtual buttons may also be utilized.

The electronic components of the glucose measurement system 100 can be disposed on, for example, a printed circuit board situated within the housing 11 and forming the data management unit 140 of the herein described system. FIG. 1B illustrates, in simplified schematic form, several of the electronic components disposed within the housing 11 for purposes of this embodiment. The data management unit 140 includes a processing unit 122 in the form of a microprocessor, a microcontroller, an application specific integrated circuit (“ASIC”), a mixed signal processor (“MSP”), a field programmable gate array (“FPGA”), or a combination thereof, and is electrically connected to various electronic modules included on, or connected to, the printed circuit board, as will be described below. The processing unit 122 is electrically connected to, for example, a test strip port circuit 104 via a communication line 123. The strip port circuit 104 is electrically connected to a strip port connector 22 during blood glucose testing. To measure analyte concentration, the strip port circuit 104 detects a resistance across electrodes of analyte test strip 24 having a blood sample disposed thereon, using a potentiostat, and converts an electric current measurement into digital form for presentation on the display 14. The processing unit 122 can be configured to receive input from the strip port circuit 104 and may also perform a portion of the potentiostat function and the current measurement function. The analyte test strip 24 can be in the form of an electrochemical glucose test strip. The test strip 24 can include one or more working electrodes. Test strip 24 can also include a plurality of electrical contact pads, where each electrode can be in electrical communication with at least one electrical contact pad. Strip port connector 22 can be configured to electrically interface to the electrical contact pads and form electrical communication with the electrodes. Test strip 24 can include a reagent layer that is disposed over at least one electrode. The reagent layer can include an enzyme and a mediator. Exemplary enzymes suitable for use in the reagent layer include glucose oxidase, glucose dehydrogcnase (with pyrroloquinoline quinone co-factor, “PQQ”), and glucose dehydrogenase (with flavin adenine dinucleotide co-factor, “FAD”). An exemplary mediator suitable for use in the reagent layer includes ferricyanide, which in this case is in the oxidized form. The reagent layer can be configured to physically transform glucose into an enzymatic by-product and in the process generate an amount of reduced mediator (e.g., ferrocyanide) that is proportional to the glucose concentration. The working electrode can then be used to measure a concentration of the reduced mediator in the form of a current. In turn, strip port circuit 104 can convert the current magnitude into a glucose concentration.

A display module 119, which may include a display processor and display buffer, is electrically connected to the processing unit 122 over the communication line 123 for receiving and displaying output data, and for displaying user interface input options under control of processing unit 122. The structure of the user interface, such as menu options, is stored in user interface module 103 and is accessible by processing unit 122 for presenting menu options to a user of the blood glucose measurement system 100. An audio module 120 includes a speaker 121 for outputting audio data received or stored by the DMU 140. Audio outputs can include, for example, notifications, reminders, and alarms, or may include audio data to be replayed in conjunction with display data presented on the display 14. For example, stored audio data may include voice data which, when replayed over speaker 121, can be heard by the user to state “Insert test strip now” or “Remove test strip now”, and similar helpful instructions, or other information. Such stored audio data can be accessed by processing unit 122 and executed as playback data at appropriate times. A volume of the audio output is controlled by the processing unit 122, and the volume setting can be stored in settings module 105, as determined by the processor or as adjusted by the user. Although not shown, the audio module 120 may be connected to a vibration motor for outputting a reminder in the form of a vibration or to otherwise notify the user when the audio is turned off. User input module 102 receives inputs via user interface buttons 16, 18, and 20 which are processed and transmitted to the processing unit 122 over the communication line 123. Although not shown in FIG. 1B, the processing unit 122 may have electrical access to a digital time-of-day clock connected to the printed circuit board for recording dates and times of blood glucose measurements, which may then be accessed, uploaded, or displayed at a later time as necessary. Associated with the clock is a timer for recording elapsed times under programmed control of the processing unit 122. Also not shown in FIG. 1B is a counter, accessible by the processing unit 122, that counts a number of blood glucose tests performed by the analyte meter 10. The counter can be monitored by the processing unit 122 to determine if a supply of glucose test strips 24 carried in the housing 11 has been depleted to a predetermined threshold amount. If so, the processing unit 122 may be programmed to issue a visual or audible notification, or both, to a user of the analyte meter 10. The counter may be reset by the user when a fresh supply of test strips is stored in the housing 11.

The display 14 can alternatively include a backlight and, as mentioned above, the strip port may include an illumination panel 12. The brightness of the display backlight and the illumination panel may be controlled by the processing unit 122 via a light source control module 115. The illumination panel 12 may be made of clear plastic and illuminated from within housing 11 by, for example, an LED light source. Similarly, the user interface buttons 16, 18, 20 may also be illuminated using LED light sources electrically connected to processing unit 122 for controlling a light output of the buttons. The light source module 115 is electrically connected to the display backlight, the illumination panel 12 and processing unit 122. Default brightness settings of all light sources, as well as settings adjusted by the user, are stored in a settings module 105, which is accessible and adjustable by the processing unit 122.

A memory module 101, that includes but are not limited to volatile random access memory (“RAM”) 112, a non-volatile memory 113, which may comprise read only memory (“ROM”) or flash memory, and a circuit 114 for connecting to an external portable memory device via a data port 13, is electrically connected to the processing unit 122 over a communication line 123. External memory devices may include flash memory devices housed in thumb drives, portable hard disk drives, data cards, or any other form of electronic storage devices. The on-board memory can include various embedded applications executed by the processing unit 122 for operation of the analyte meter 10, as will be explained below. On board memory can also be used to store a history of a user's blood glucose measurements including dates and times associated therewith. Using the wireless transmission capability of the analyte meter 10 or the data port 13, as described below, such measurement data can be transferred via wired or wireless transmission to connected computers or other processing devices.

A wireless module 106 may include transceiver circuits for wireless digital data transmission and reception via one or more internal digital antennas 107, and is electrically connected to the processing unit 122 over communication line 123. The wireless transceiver circuits may be in the form of integrated circuit chips, chipsets, programmable functions operable via processing unit 122, or a combination thereof. Each of the wireless transceiver circuits is compatible with a different wireless transmission standard. For example, a wireless transceiver circuit 108 may be compatible with the Wireless Local Area Network IEEE 802.11 standard known as WiFi. Transceiver circuit 108 is configured to detect a WiFi access point in proximity to the analyte meter 10 and to transmit and receive data from such a detected WiFi access point. A wireless transceiver circuit 109 may be compatible with the Bluetooth protocol and is configured to detect and process data transmitted from a Bluetooth “beacon” in proximity to the analyte meter 10. A wireless transceiver circuit 110 may be compatible with the near field communication (“NFC”) standard and is configured to establish radio communication with, for example, an NFC compliant point of sale terminal at a retail merchant in proximity to the analyte meter 10. A wireless transceiver circuit 111 may comprise a circuit for cellular communication with cellular networks and is configured to detect and link to available cellular communication towers.

A power supply module 116 is electrically connected to all modules in the housing 11 and to the processing unit 122 to supply electric power thereto. The power supply module 116 may comprise standard or rechargeable batteries 118 or an AC power supply 117 may be activated when the analyte meter 10 is connected to a source of AC power. The power supply module 116 is also electrically connected to processing unit 122 over the communication line 123 such that processing unit 122 can monitor a power level remaining in a battery power mode of the power supply module 116.

In addition to connecting external storage for use by the analyte meter 10, the data port 13 can be used to accept a suitable connector attached to a connecting lead, thereby allowing the analyte meter 10 to be wired to an external device such as a personal computer. Data port 13 can be any port that allows for transmission of data such as, example, a serial, USB, or a parallel port.

In terms of operation, one aspect of the analyte meter 10 may include a capability for automatically adjusting features of the blood glucose measurement system 100 based on its surroundings. For example, a user of the analyte meter 10 may be in a location that is typically noisy, such as an airport terminal, a bus or train station or a shopping mall. Previously, the user may have performed a blood glucose test in a quiet meeting room at work, and so had turned off the audio reminder so as not to draw attention to himself or herself during a meeting. In the exemplary embodiment, the audio will remain off until the WiFi module detects, for example, a “JFK terminal 1” or other WiFi network that is associated by way of a stored rule set or table. Upon association, the DMU 140 is configured to automatically set the audio output of the analyte meter 10 to the loudest level.

Another situation may take place, for example, in a poorly lit restaurant. The user enters the restaurant to eat a meal, and so performs a blood glucose test. As soon as the user approaches the restaurant, its Bluetooth network transmits a beacon frame to advertise offers to passers by having mobile smart phones. The beacon is detected by the analyte meter 10, which continues detecting the Bluetooth beacon after some time has passed and determines that the user is not merely passing by but has remained in range of the restaurant for several minutes, therefore is probably intending to eat and therefore will probably want to perform a blood glucose test. The analyte meter 10 recognizes the restaurant name after detecting and processing the Bluetooth beacon frame data and sets the display backlight brightness high and the audio off. The user is then able to perform a blood test discreetly. Similarly, a user may be in a similarly and poorly lit movie theater while the analyte meter 10 has already detected the “Cinema” WiFi beacon and sets the audio off and the display backlight brightness and strip port illumination to low so as not to draw attention in the darkened theater when performing a blood glucose test. The analyte meter 10 can be similarly configured for other locales, such as the user's own home when it detects the user's home WiFi network, for example. The time-of-day clock can be accessed by the processing unit 122 to adjust audio and light output levels in response to particular times of day, thereby providing further control refinements over the operation of the blood glucose measurement system 100.

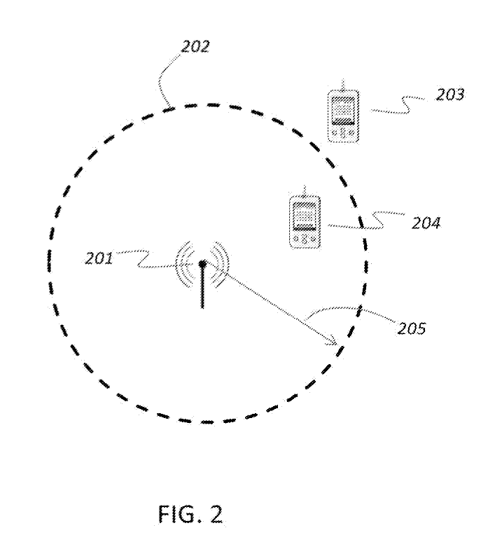

The automatically adjusted settings just described are accomplished by the analyte meter 10, and more particularly the DMU 140, as follows. With reference to FIG. 2, there is illustrated a wireless access point, or WLAN, provided by Wi-Fi antenna 201 that can transmit and receive wireless data over a certain radial distance 205 resulting in a coverage area 202. Wireless device 204, which may be, for example, the analyte meter 10, has been carried into coverage area 202 by a user, and can therefore establish bidirectional (i.e., two-way) radio communication with access point 201. Wireless access device 203, for example, is outside the coverage area 202 of wireless access point 201 and so cannot detect transmissions from wireless access point 201 or establish a bi-directional radio communication channel therewith.

Wireless access points, such as 201, are increasingly deployed in various public locations such as coffee shops, restaurants, movie theaters, shopping malls, hotels, parks, museums, airports, and the like. Standard WiFi transmissions from access point 201 include broadcast of identification data commonly known as a service set identifier (“SSID”) which identifies the particular wireless access point. The identifier includes an alphanumeric character string, i.e. text or, that is commonly used by wireless communication devices to notify a user that a wireless access point is in proximity to the user and is available for two way communication, which typically includes network access to the internet, for example. The character string typically identifies a commercial establishment that is providing the wireless access point by a trade name recognizable to most users if the establishment is well known. The name of the establishment typically appears in a prompt presented to the user on a display screen of the wireless communication device asking the user if he or she wishes to establish network communication using the wireless access point. In order to display the prompt, the wireless transmission device extracts the alphanumeric character string from the broadcast identification data to be presented to the user.

DMU 140 makes use of such identifiers extracted from WiFi access points, such as access point 201, in order to automatically adjust settings of various features provided by the glucose measurement system 100 during use based on a rules set. In one aspect, the contained DMU 140 stores a table of retail establishments by trade name, such as names of coffee shops, restaurants, movie theaters, shopping malls, hotels, parks, museums, airports, and the like. The table may be stored in the memory module 101 or in a memory of settings module 105. Associated with each such table entry according to the exemplary embodiment is settings information that may be accessed by the processing unit 122 and stored in settings module 105 whereby various features of the analyte meter 10 can be adjusted according to the settings information. Various other information may be stored in the table in association with each table entry, such as descriptions of the type of merchandise available in the establishments that provide the wireless access point. The device settings information can include one or more adjustments, for example, a brightness of the display 14 backlight, a brightness of the LED that illuminates illumination panel 12 or the LEDs that illuminate buttons 16, 18, 20, a volume level of alarms, reminders, and notifications played on speaker 121, or a combination thereof. The information describing the type of establishment might identify the wireless access point provider as a restaurant, a grocer, a drugstore, or the like. Such a table can be preloaded during manufacture of the analyte meter 10 or it can be populated by accessing a web site of a company such as iPass, which is a commercial internet company that provides downloadable current information regarding a large number of WiFi access point providers.

An exemplary table that is accessed by processing unit 122 is shown below. The first column lists alphanumeric character strings that are typically embedded in the SSID as transmitted by a wireless access point provider, such as by wireless access point 201. Settings and descriptions are associated with each particular named wireless access provider by storing relevant information in succeeding columns of the same row as the named provider. Thus, the processing unit 122 can retrieve settings information associated with an identified provider, such as by reading a Backlight brightness level from column 2 and an Audio volume level from column 3, and storing those numerical level data in settings module 105, whereby, in response to the setting information stored therein, various features of the analyte meter are adjusted.

| Character String | Settings | Settings | | |
|---|---|---|---|---|
| Identifier (SSID) | Backlight | Audio | . . . | Description |
| Starbuck's | Level 8 | Level 4 | . . . | Coffee Shop |
| Denny's | Level 8 | Level 4 | . . . | Restaurant |
| . . . | . . . | . . . | . . . | . . . |
| Walgreen's | [Do not | [Do not | . . . | Drugstore |
| | Adjust] | Adjust] | | |
| Regal | Level 3 | Level 2 | . . . | Movie Theater |
| JFK Terminal | Level 9 | Level 10 | . . . | Airport |

Similar to the WiFi access point communication method just described, a Bluetooth transmission also includes data for identifying a source of the transmitted Bluetooth beacon frames and can be similarly detected and used by DMU 140 to access stored settings and adjust features of the glucose measurement system 100 corresponding thereto. Similar to both the WiFi access point and Bluetooth communication methods just described, an NFC transmission also includes data for identifying a source of the NFC signals and can be detected and used by DMU 140 to access stored settings and adjust features corresponding thereto.

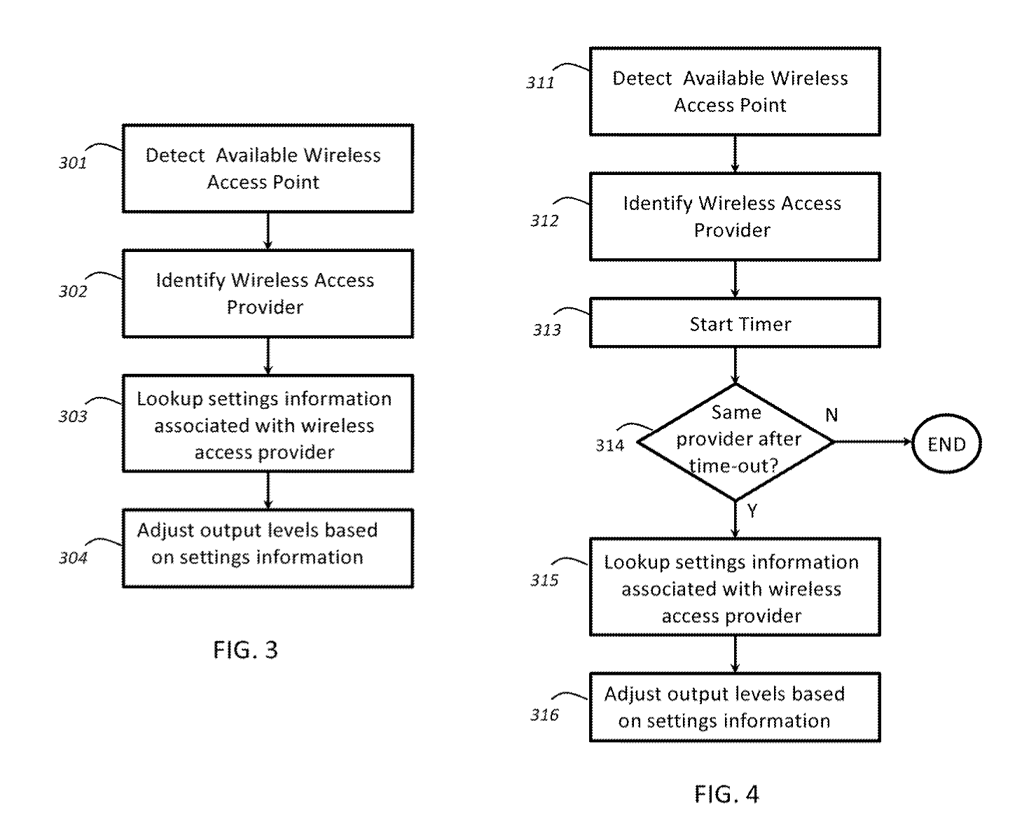

With reference to FIG. 3, there is illustrated a programmed method of operating the glucose measurement system 100 that utilizes several of its features just described. The DMU 140 may include programs stored in memory module 101 accessible by processing unit 122. Under program control, processing unit 122 constantly monitors incoming wireless signals for wireless transmissions from wireless access points in proximity to the glucose measurement system 100. At step 301, analyte meter 10 may be carried by a user into a coverage area of wireless access point and detects, via antenna 107, standard transmissions broadcast by the wireless access point. The wireless access point may comprise a WiFi access point, a Bluetooth access point, an NEC access point, or any other data transmission that includes but are not limited to identification data of a source of the data transmission. At step 302, processing unit 122 automatically identifies a source, or provider, of the wireless access point from standard identification data transmitted therefrom and stores the identification data. At step 303, processing unit 122 accesses a table or database stored therein, using the alphanumeric identifier extracted from the standard identification data. A table lookup accesses information associated with the extracted identifier and retrieves settings information for adjusting one or more features of the analyte meter 10. At step 304, the processing unit 122 stores the settings information in settings module 105 which triggers an adjustment, if any, of one or more feature settings provided by DMU 140 during, for example, a blood glucose test.

With reference to FIG. 4, there is illustrated another programmed method of operating the analyte meter 10 that utilizes several of its features, described above, via programs stored in memory module 101 as accessed by processing unit 122. As described above, under program control, processing unit 122 constantly monitors incoming wireless signals for wireless transmissions from wireless access points in proximity to the analyte meter 10. At step 311, the analyte meter 10 may be carried by a user into a coverage area of wireless access point, such as wireless access point 201 (FIG. 2), and detects, via antenna 107, standard transmissions broadcast by the wireless access point. The wireless access point may comprise a WiFi access point, a Bluetooth access point, an NFC access point, or any other data transmission that includes but are not limited to identification data of a source of the data transmission. At step 312, processing unit 122 automatically identifies a source, or provider, of the wireless access point from standard identification data transmitted therefrom and stores the identification data. At step 313, processing unit starts a timer that is preset for a selected adjustable duration stored in memory module 101. After the timer times out at the end of the selected duration, at step 314, processing unit 122 checks whether the same wireless access point is still detected in proximity to glucose measurement system 100 using the identification data, previously stored, for comparison. If it's not still detected, the program portion terminates and processing unit continues monitoring incoming wireless signals as before. If the same wireless provider is still detected at step 314 then, at step 315, processing unit 122 accesses a table or database stored therein, using the alphanumeric identifier extracted from the standard identification data. A table lookup accesses information associated with the extracted identifier and retrieves settings information for adjusting one or more features. At step 316, the processing unit 122 stores the settings information in settings module 105 which triggers an adjustment, if any, of one or more feature settings provided by DMU 140 during, for example, a blood glucose test.

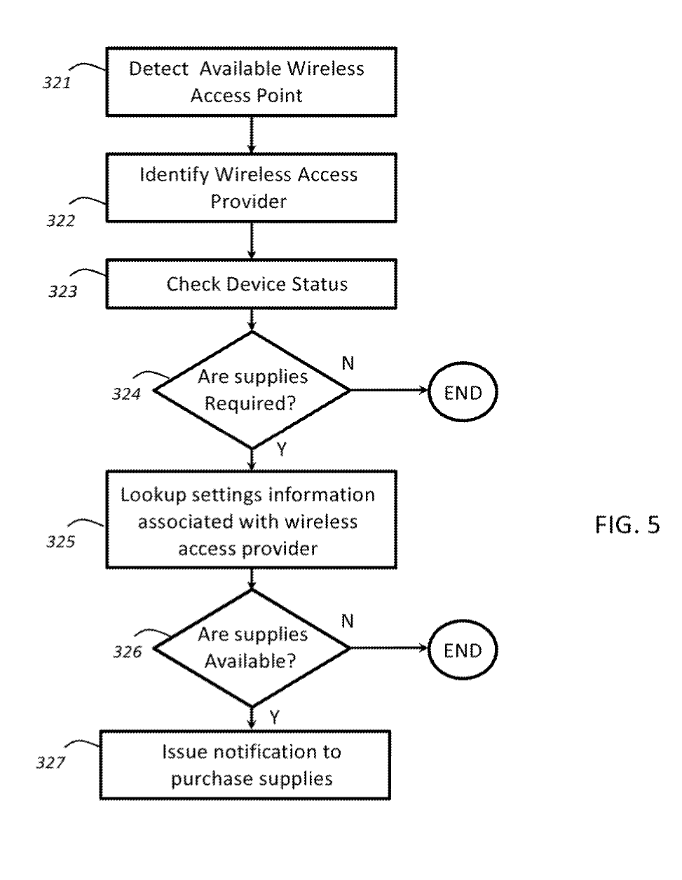

With reference to FIG. 5, there is illustrated another programmed method of operating analyte meter 10 that utilizes several of its features, described above, via programs stored in memory module 101 as accessed by processing unit 122. As described above, under program control, processing unit 122 constantly monitors incoming wireless signals for wireless transmissions from wireless access points in proximity to the analyte meter 10. At step 321, the analyte meter 10 may be carried by a user into a coverage area of wireless access point, such as wireless access point 201 (FIG. 2), and detects, via antenna 107, standard transmissions broadcast by the wireless access point. The wireless access point may comprise a WiFi access point, a Bluetooth access point, an NFC access point, or any other data transmission that may include but are not limited to identification data of a source of the data transmission. At step 322, processing unit 122 automatically identifies a source, or provider, of the wireless access point from standard identification data transmitted therefrom and stores the identification data. At step 323, processing unit checks device status, such as one or more of a battery power level or a count of how many blood glucose tests have been performed since a supply of glucose test strips was last replenished or since the counter was last reset. At step 324, processing unit 122 determines whether supplies are required, such as fresh batteries or a supply of glucose test strips 24. If they are not required, the program portion terminates and processing unit continues monitoring incoming wireless signals as before. If supplies are required as determined at step 324 then, at step 325, processing unit 122 accesses a table or database stored therein, using the alphanumeric identifier extracted from the standard identification data. A table lookup accesses information associated with the extracted identifier and retrieves information describing the provider of the detected wireless access point. At step 326, the processing unit determines if the provider of the wireless access point is a merchant of any of the required supplies. If the provider is not such a merchant, the program portion terminates and processing unit continues monitoring incoming wireless signals as before. If the wireless access point provider is determined to be a merchant of the required supplies, such as a drugstore, then, at step 327, the processing unit 122 issues an audible or visual notification, or both, that a provider of such supplies is in proximity to the DMU 140.

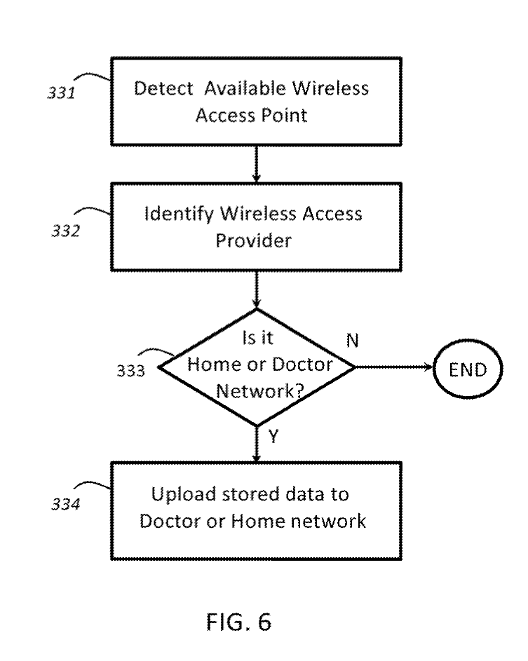

With reference to FIG. 6, there is illustrated another programmed method of operating the analyte meter 10 that utilizes several of its features, described above, via programs stored in memory module 101 as accessed by processing unit 122. As described above, under program control, processing unit 122 constantly monitors incoming wireless signals for wireless transmissions from wireless access points in proximity to the analyte meter 10. At step 331, the analyte meter 10 may be carried by a user into a coverage area of wireless access point, such as wireless access point 201 (FIG. 2), and detects, via antenna 107, standard transmissions broadcast by the wireless access point. The wireless access point may comprise a WiFi access point, a Bluetooth access point, an NFC access point, or any other data transmission that includes but are not limited to identification data of a source of the data transmission. At step 332, processing unit 122 automatically identifies a source, or provider, of the wireless access point from standard identification data transmitted therefrom and stores the identification data. At step 333, processing unit checks whether the identified wireless access point is the user's home network or a wireless access point identified as the user's doctor's network provided, for example, by a WLAN at the doctor's office. Typically, data identifying the user's WiFi home network Of the doctor's WiFi network is entered by the user into the table for storing WiFi provider identification data. When an unknown WiFi access point is first detected by the processing unit 122, a default program may be executed wherein the user is prompted to enter data identifying the wireless access point, for example, “Home” or “Doctor”. Thereafter, when wireless access is detected, this access point can be identified in accordance therewith. If, at step 333, the processing unit 122 determines that the identified network is not the “Home” wireless access point or the “Doctor” wireless access point, the program portion terminates and the processing unit 122 continues monitoring incoming wireless signals as before. If, at step 333, the wireless access point is determined to be the user's home access point or the user's doctor's wireless access point, then, at step, 334, the processing unit 122 automatically initiates an upload of blood glucose test data stored in a memory of the memory module 101.

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.), or an embodiment combining software and hardware aspects that may all generally be referred to herein as a “circuit,” “circuitry,” “module,” and/or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible, non-transitory medium that can contain, or store a program for use by or in connection with an instruction execution system, apparatus, or device.

Program code and/or executable instructions embodied on a computer readable medium may be transmitted using any appropriate medium, including but not limited to wireless, wireline, optical fiber cable, RE, etc., or any suitable combination of the foregoing.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Furthermore, the various methods described herein can be used to generate software codes using off-the-shelf software development tools such as, for example, Visual Studio 6.0, C or C++ (and its variants), Windows 2000 Server, and SQL Server 2000. The methods, however, may be transformed into other software languages depending on the requirements and the availability of new software languages for coding the methods.

### PARTS LIST FOR FIGS. 1A-6

10 analyte meter
11 housing, meter
12 strip port illumination panel
13 data port
14 display
16 user interface button
17 first marking
18 user interface button
19 second marking
20 user interface button
21 third marking
22 strip port connector
24 glucose test strip
100 blood glucose measurement system
101 memory module
102 buttons module
103 user interface module
104 strip port module
105 DMU settings module
106 transceiver module
107 antenna
108 WiFi module
109 Bluetooth module
110 NFC module
111 GSM module
112 RAM module
113 ROM module
114 external storage
115 light source module
116 power supply module
117 AC power supply
118 battery power supply
119 display module
120 audio module
121 speaker
122 processing unit
123 communication line
140 data management unit (DMU)
200 wireless access area
201 wireless access point
202 wireless access coverage area
203 device beyond wireless reach
204 device within wireless reach
205 radius of wireless reach
301 step—detect wireless access point
302 step—identify wireless provider
303 step—lookup settings information
304 step—adjust output levels
311 step—detect wireless access point
312 step—identify wireless provider
313 step—start timer
314 decision step—same provider after time-out?
315 step—lookup settings information
316 step—adjust output levels
321 step—detect wireless access point
322 step—identify wireless provider
323 step—check device status
324 decision step—are supplies required?
325 step—lookup settings information
326 decision step—are supplies available?
327 step—issue notification
331 step—detect wireless access point
332 step—identify wireless provider
333 decision step—home or doctor network?
334 step—upload stored data to network

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

What is claimed is:

1. An automated method of operating a wireless blood glucose measurement system having an analyte meter, said measurement system including a data management unit having a microprocessor, a memory, and a wireless communication protocol, the method comprising the steps of:
detecting a wireless communication channel in proximity to the analyte meter;
said data management unit automatically identifying a source of the detected wireless communication channel; and
automatically adjusting at least one feature relating to said measurement system for enabling use of said analyte meter based on the identity of said source.

2. The method of claim 1, wherein the step of automatically adjusting is further based on an amount of time that the source is detected.

3. The method of claim 1, wherein the step of automatically adjusting includes the further step of accessing a table stored in the memory that lists at least one feature adjustment corresponding to the identity of the source.

4. The method of claim 3, further comprising generating and storing the table in the memory during manufacture of the blood glucose measurement system.

5. The method of claim 1, further comprising performing a blood glucose test using the analyte meter and the at least one feature that has been automatically adjusted.

6. The method of claim 5, wherein the step of automatically adjusting includes the further step of increasing or decreasing a light level of the analyte meter.

7. The method of claim 5, wherein the step of automatically adjusting includes the further step of increasing or decreasing a volume level of an audible notification indicating that a blood glucose test is required.

8. The method of claim 1, further comprising:
automatically determining a number of test strips remaining in the analyte meter; and
the data management unit automatically outputting a visual notification on a display of said analyte meter, an audible notification through a speaker of said analyte meter, or both, if the data management unit determines that the source comprises a merchant of the test strips.

9. The method of claim 1, further comprising:
automatically determining a battery power level of the analyte meter; and
automatically outputting a visual notification on a display of said analyte meter, an audible notification through a speaker of said meter, or both, if the data management unit determines that the source comprises a merchant of the batteries.

10. The method of claim 1, further comprising:
storing a result of a blood glucose test in a memory of the data management unit; and
automatically uploading the stored result of the blood glucose test to a computer system providing the detected wireless communication channel.

11. An analyte meter comprising:
a wireless communication circuit for receiving wireless communication transmitted from a nearby wireless communication source wherein the wireless communication includes a character string identifying a source of the wireless communication;
a preloaded electronic table comprising a plurality of character strings each stored in association with information describing an identity of a corresponding wireless communication source; and
a data management unit comprising a circuit for adjusting at least one of a visual or audio output level of the analyte meter based on the identity of the communication source.

12. The analyte meter of claim 11, further comprising a circuit for maintaining the adjusted visual or audio output level while the analyte meter is performing a blood glucose test.

13. The analyte meter of claim 11, wherein the data management unit comprises a timer for delaying said adjusting the visual or audio output level for a preselected duration so long as the wireless communication circuit continues to receive the wireless communication after the preselected duration.

14. The analyte meter of claim 11, wherein the data management unit comprises a circuit for reducing a light level of a test strip port, user interface buttons, a backlight of a display screen, or a combination thereof.

15. The analyte meter of claim 11, wherein the data management unit further comprises:
a counter for counting a number of blood glucose tests performed by the analyte meter;
a circuit for automatically accessing the electronic table to determine if the information describing the communication source indicates that the communication source comprises a merchant of test strips used for the blood glucose tests; and
a circuit connected to both a display for outputting a visual notification and to a speaker for outputting an audible notification in response to the circuit for automatically accessing the electronic table determining that the communication source comprises a merchant of the test strips.

16. The analyte meter of claim 11, wherein the data management unit further comprises:
a battery power level circuit for measuring a power level of a battery that provides electric power to the analyte meter;
a circuit for automatically accessing the electronic table to determine if the information describing the communication source indicates that the communication source comprises a battery merchant, in response to the power level circuit indicating that the power level of the battery has reached a preset threshold; and
a circuit connected to both a display for outputting a visual notification and to a speaker for outputting an audible notification in response to the circuit for automatically accessing the electronic table determining that the communication source comprises a battery merchant.

17. A method of operating a portable wireless blood glucose measurement system, the method comprising:
detecting a wireless communication channel;
determining parameters of a location of the measurement system based on the detected wireless communication channel; and
adjusting a data output level of the measurement system based on the parameters of the location.

18. The method of claim 17, further comprising storing a plurality of names of known sources of wireless communication channels, and wherein the step of detecting comprises detecting a name of the source of the wireless communication channel and comparing the name of the detected wireless communication channel with the stored plurality of names.

19. The method of claim 18, further comprising storing a plurality of output settings each associated with one of the stored plurality of names of the known sources of the wireless communication channels, and wherein the step of adjusting includes adjusting the data output level of the measurement system according to a stored output setting associated with one of the stored plurality of names that matches the name of the source of the detected wireless communication channel.

20. The method of claim 19, wherein the step of detecting comprises detecting a WiFi communication channel, a Bluetooth communication channel, or a Near Field Communication channel.
